(19) United States
(12) Patent Application Publication
(10) Pub. No.: US 2026/0241094 A1
(43) Pub. Date: Aug. 20, 2026

(54) TUBE CONNECTOR

(71) Applicant: Unomedical A/S, Lejre (DK)

(72) Inventors: Anders Hede, Gentofte (DK); Omid Mostafaei, Lejre (DK); Rachel Pytel, Newton, MA (US)

(21) Appl. No.: 19/639,282

(22) Filed: Apr. 5, 2026

Related U.S. Application Data
(63) Continuation of application No. PCT/EP2024/078067, filed on Oct. 4, 2024.
(60) Provisional application No. 63/588,323, filed on Oct. 6, 2023.

(30) Foreign Application Priority Data
Jan. 29, 2024 (GB) 2401158.7

(51) Int. Cl. A61M 5/14; A61M 5/142; A61M 5/162; A61M 5/165; A61M 39/12; A61M 39/20

(52) U.S. Cl. CPC A61M 5/1413; A61M 5/142; A61M 5/162; A61M 5/165; A61M 39/12; A61M 39/20

(57) ABSTRACT

A tube connector for an infusion hub, the tube connector comprising: a body portion comprising: a first end connectable to a tube for receiving a therapeutic agent from a pump; a second end connectable to a fluid transfer part for fluid communication with a cannula of the infusion hub, in use; an outer surface; a fluid flow path defined between the first end and the second end of the body portion; a cavity positioned between the first end and the second end of the body; and a filter, wherein the cavity comprises an opening on the outer surface of the body portion, through which the filter is inserted into the cavity.

The invention relates to a tube connector. More particularly, although not exclusively, the invention relates to a tube connector for an infusion hub, for example an infusion hub for subcutaneous delivery of a therapeutic agent into a patient and associated methods. Particularly, though not exclusively, the invention relates to an infusion set including an infusion hub for subcutaneous infusion of insulin, heparin, apomorphine, arbidopa or levodopa and/or levodopa products into a patient.

### BACKGROUND

For patients with diabetes, insulin therapy is often an important part of their treatment, helping to regulate blood sugar levels and store excess glucose for energy. There are two principal modes for delivering insulin. The first mode includes syringes and injector pens, which are used to inject a dose of insulin typically three or four times a day (depending on, inter alia, the type of diabetes and blood sugar levels of the patent). While these devices are simple and low cost, delivering each dose of insulin requires a needle stick. The second mode uses an infusion pump, sometimes called an insulin pump, which delivers controlled doses of insulin throughout the day. An infusion pump can be used to deliver insulin to a patient continuously (basal dose), on demand (bolus dose) or at scheduled intervals. Infusion pumps are more complex and expensive than syringes and pens, though enable improved regulation of blood sugar levels, for example by programmable delivery schedules, and require fewer needle sticks.

The second mode is known as continuous subcutaneous insulin infusion (CSII) therapy. Infusion pumps for CSII may be worn by the patient. The systems typically include a combined infusion pump and reservoir for containing an insulin drug, for example human insulin or analogue insulin, and an insulin infusion set. The infusion set may include a cannula (for example, a polymeric catheter or metal needle) for insertion subcutaneously into the patient and a flexible tube for fluidly connecting the cannula to the reservoir. Once the cannula is inserted into the patient, it may remain in place for a period of time, i.e. days, to allow for continuous delivery of the insulin drug. The current recommended wear time for insulin infusion sets is two to three days, to avoid problems that may arise relating to the infusion set itself or to the infusion site. However, such problems may still arise within recommended wear times, resulting in early removal of the infusion set and more frequent site rotation across infusion sites (for example buttocks, abdomen and arms).

While problems relating to the infusion set have been well investigated and addressed in recent years, there remains little understanding and few solutions to address problems relating to the infusion site. Problems relating to the infusion site include pain, bleeding, infection, skin irritation, erythema, lipohypertrophy and lipoatrophy. Problems at the infusion site may lead to the build-up of scar tissue, which consequently lowers insulin sensitivity and increases the risk of hypoglycaemia, as well as having a cosmetic impact on patients. All these problems can deter patients from continuing to use their insulin pumps, resulting in poorer patient outcomes.

It is known that problems at the infusion site are a consequence of the immune response to the presence of the cannula and the insulin drug in the body. The immune system responds by activating and progressing the foreign body reaction (FBR)—an inflammatory and fibrotic process that occurs upon introducing a foreign material into the body. In FBR, cells of the immune system identify foreign materials (such as unwanted biological, chemical, or physical species, present in infusible solutions) and attempt to degrade it, or otherwise encapsulate the material by forming a physical barrier to isolate it from the rest of the body. FBR is a problem for increasing wear times of infusion sets in CSII therapy, as the immune system reacts to the inserted cannula and the insulin drug. This limitation prevents realizing the full potential of CSII therapy.

Generally, unwanted species, whether biological, chemical or physical, present in infusible solutions have undesirable consequences for patients.

It is an object of embodiments of the invention to provide an improved tube connector for an infusion hub that attempts to circumvent FBR, increase wear times of infusion sets, and/or at least mitigate one or more problems associated with known arrangements.

### BRIEF SUMMARY

The invention is defined by the appended claims.

According to an aspect of the invention there is provided a tube connector for an infusion hub. The tube connector may be configured to engage a fluid transfer part of the infusion hub. The tube connector may be connectable to a tube for receiving a therapeutic agent from a pump.

According to an aspect of the invention there is provided an infusion set including an infusion hub, the infusion hub including a casing, a cannula for insertion into a patient, a fluid transfer part connected to the casing and in fluid communication with the cannula and a tube connector.

The tube connector may be configured to engage the fluid transfer part and be connectable to a tube for receiving a therapeutic agent from a pump.

The infusion set may be part of an infusion system.

According to another aspect of the invention there is provided an infusion system including an infusion set and a source of medicament for delivery to the patient via the tube connector and the cannula.

The infusion system may include a pump for continuous or intermittent delivery of medicament to the patient.

According to an aspect of the invention there is provided a tube connector, for example a tube connector for an infusion hub. The tube connector may include a body portion including a first end connectable to a tube for receiving a therapeutic agent from a pump and a second end connectable to a fluid transfer part for fluid communication with a cannula of the infusion hub, in use. The body portion may include a fluid flow path. The fluid flow path may be defined between the first end and the second end of the body portion. The body portion may include a cavity positioned between the first end and the second end of the body. The body portion may include a filter. The filter may be located within the cavity.

According to an aspect of the invention there is provided a tube connector for an infusion hub, the tube connector including a body portion. The body portion includes a first end connectable to a tube for receiving a therapeutic agent from a pump, a second end connectable to a fluid transfer part for fluid communication with a cannula of the infusion hub, in use. The body portion includes a fluid flow path defined between the first end and the second end of the body portion, a cavity positioned between the first end and the second end of the body, and a filter, wherein the filter is located within the cavity.

According to an aspect of the invention, there is provided a tube connector for an infusion hub, the tube connector including: a body portion including: a first end connectable to a tube for receiving a therapeutic agent from a pump; a second end connectable to a fluid transfer part for fluid communication with a cannula of the infusion hub, in use; an outer surface; a fluid flow path defined between the first end and the second end of the body portion; a cavity positioned between the first end and the second end of the body; and a filter, wherein the cavity includes an opening on the outer surface of the body portion, and wherein the opening is configured to receive the filter. The filter may be, for example, inserted into the cavity via the opening, in use.

A central axis may extend between the first end and the second end of the body portion. The fluid flow path may extend along the central axis. The fluid flow path may have a central axis that is coaxial with the central axis of the body portion and/or the tube connector.

The cavity may have a cavity axis. The cavity axis may be at an angle, for example a non-zero, angle to the central axis of the body portion. The cavity axis may be at an acute angle or an obtuse angle to the central axis of the body portion. The cavity axis may be perpendicular to the central axis of the body portion. In other words an angle of approximately 90 degrees may be formed between the central axis of the body portion and the cavity axis.

The body portion may include a first body portion part and a second body portion part. The cavity may be defined between the first body portion part and the second body portion part. The first body portion part and/or the second body portion part may include a hollow. When the first body portion part and the second body portion part are brought together to form the body portion, the hollow of the or each body portion part may define the cavity.

The first body portion part may form an upper portion of the body portion and the second body portion part may form a lower portion of the body portion.

Alternatively, the first body portion part may form a lower portion of the body portion and the second body portion part may form an upper portion of the body portion.

The opening may be provided in the upper portion of the body portion. The opening may define a channel. The channel defined by the opening may thus extend through the upper portion of the body portion to the cavity. In other words, the cavity may be connected to the opening through the upper portion of the body portion by the channel.

Alternatively, the opening may be provided in the lower portion of the body portion. The channel defined by the opening may thus extend through the lower portion of the body portion to the cavity. In other words, the cavity may be connected to the opening through the lower portion of the body portion by the channel.

It will thus be understood that the cavity may be positioned adjacent and connected to an opening in an outer surface (of either of the upper portion or the lower portion) of the body portion.

The first body portion part and the second body portion part may sealingly engage with each other to form the body portion.

The upper portion and the lower portion may sealingly engage with each other to form the body portion.

The body portion may include a cap that is configured to close the opening, thereby securing the filter in the cavity. The cap may sealingly close the opening. The cap may be press-fitted into the channel, thereby closing the opening.

A fluid chamber may be provided between the first end of the body portion and the cavity.

According to an aspect of the invention, there is provided a tube connector for an infusion hub, the tube connector comprising: a body portion comprising: a first end connectable to a tube for receiving a therapeutic agent from a pump; a second end connectable to a fluid transfer part for fluid communication with a cannula of the infusion hub, in use; an outer surface; a fluid flow path defined between the first end and the second end of the body portion; a cavity positioned between the first end and the second end of the body; a filter; and a fluid chamber, wherein the fluid chamber is provided between the first end of the body portion and the cavity and wherein the cavity comprises an opening on the outer surface of the body portion through which the filter is inserted into the cavity.

The fluid chamber may be a first fluid chamber. A second fluid chamber may be provided between the cavity and the second end of the body portion.

The cavity may have a depth (i.e. a cavity depth). The filter may have a depth (i.e. a filter depth). The filter depth may be greater than the cavity depth.

The cavity may have a width (i.e. a cavity width). The cavity width may be a cavity diameter in embodiments for which the cavity has a generally circular cross-section. The filter may have a width (i.e. a filter width). The filter width may be a cavity diameter in embodiments for which the filter has a generally circular cross-section.

The filter may include cellulose. The filter may include, for example, regenerated cellulose or cellulose acetate.

The filter may include one or more of polytetrafluoroethylene, nylon, polyethersulfone, polyvinylidene fluoride, polyethylene and polypropylene.

The filter may be a sintered filter.

According to an aspect of the invention there is provided an infusion set. The infusion set may include an infusion hub having a cannular and a mounting portion. The mounting portion may be suitable for mounting the infusion hub to a patient's skin with the cannular in a subcutaneous position. The infusion set may also include a tube connector according to the preceding aspects of the invention.

One of the tube connector and the infusion hub may comprise a needle.

According to an aspect of the invention there is provided an infusion set including an infusion hub having a cannula and a mounting portion for mounting the infusion hub to a patient's skin with the cannula in a subcutaneous position, and the tube connector of any one of the preceding aspects of the invention.

One of the tube connector and the infusion hub may include a needle.

According to an aspect of the invention there is provided an infusion system. The infusion system may include an infusion set according to any of the preceding aspects of the invention. The infusion system may include a source of medicament for delivery to the patient via the tube connector and the cannula. The infusion system may include a pump. The pump may be for continuous or intermittent delivery of medicament to the patient.

According to an aspect of the invention there is provided an infusion system including the infusion set of the preceding aspects of the invention and a source of medicament for delivery to the patient via the tube connector and the cannula.

The infusion system may include a pump for continuous or intermittent delivery of medicament to the patient.

Tube connectors with filters as described herein may be useful in inhibiting FBR at an infusion site, and thereby may avoid problematic occurrences such as coagulation, occlusion and/or inflammation at the infusion site and/or encapsulation of the cannula. In particular, devices as described herein may be useful in inhibiting FBR at the infusion site in diabetic patients receiving CSII therapy. The infusion site may be a single infusion site in use for an extended period of time, for example at least four days.

Tube connectors with filters as described herein may be useful in removing unwanted species from a therapeutic agent before delivery of the therapeutic agent to a patient, for example where unwanted species include preservatives necessarily present in insulin solutions to stabilize and/or sterilize insulin solutions prior to delivery to a patient, but which are cytotoxic.

The provision of an opening in the outer surface of the body portion enables the size of filters used with the tube connector to be increased. Larger filters have a greater filter area. Greater filter areas beneficially preserve an unrestricted flow of therapeutic agent through the tube connector.

The provision of a cavity, with or without fluid chambers, allows the therapeutic agent to disperse over a larger area prior to passing through the filter.

Axial mounting of the insert within the body portion allows the requirements of a tube coupling and mounting a filter to be de-coupled, allowing the filter properties to be optimized.

### BRIEF DESCRIPTION OF THE DRAWINGS

Exemplary infusion sets and tube connectors for such infusion sets are further described hereinafter with reference to the accompanying drawings, in which:

FIG. 1A is an illustration of an infusion set;

FIG. 1B is a further illustration of the infusion set of FIG. 1A;

FIG. 2A is a perspective view of a tube connector according to an embodiment of the invention;

FIG. 2B is an exploded view of the tube connector of FIG. 2A;

FIG. 2C is a cross-section view of the tube connector of FIG. 2A; and

FIG. 2D is an alternative cross-section view of the tube connector of FIG. 2A.

### DETAILED DESCRIPTION

The presently described infusion sets and tube connectors therefor have particular application for use with infusion pump systems, such as an infusion pump for delivery of a therapeutic agent, such as insulin, heparin or any other liquid therapeutic agents, where the infusion pump includes a fluid pump and a reservoir, and an infusion set having a cannula (typically part of an infusion hub) and tube for connecting the cannula to the reservoir. The infusion pump may be an insulin pump for CSII therapy, and the therapeutic agent may be an insulin formulation. The presently described infusion sets and tube connectors are able to deliver insulin to a patient at a single infusion site over an extended period of time. An extended period of time is to be understood to mean at least four days. More specifically, an extended period of time may include four to seven days, seven or more days, seven to ten days, and ten or more days. An extended period of time may include fourteen or more days.

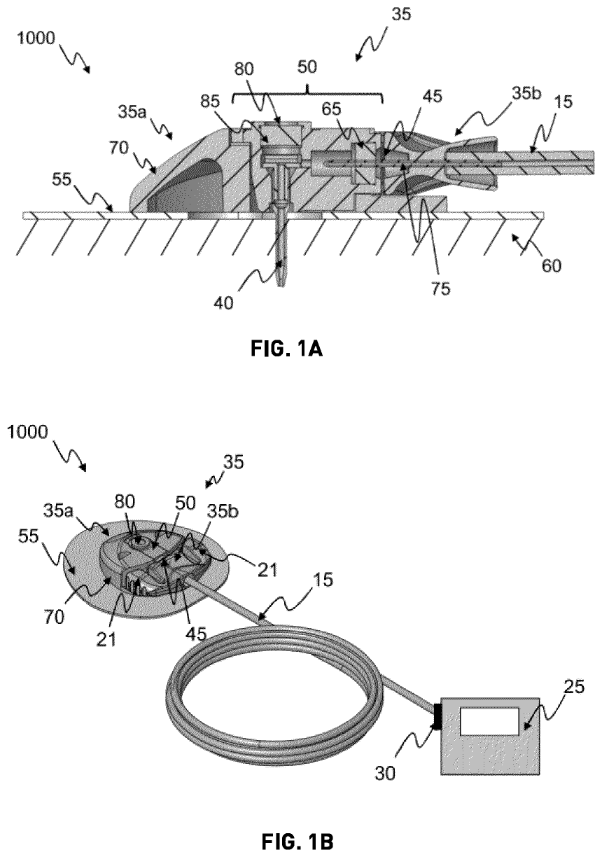

FIG. 1A and FIG. 1B illustrate an infusion set 1000. The infusion set 1000 includes an infusion hub 35, including a body 35a and a tube connector 35b. The infusion hub 35 is secured to the skin of a patient by an adhesive patch 55, which maintains a cannula 40 subcutaneously within the sub-dermal fatty tissue of patient. Tube 15 connects a pump outlet connector 30 of a pump 25 (the pump 25 containing the therapeutic agent) with the infusion hub 35 via the tube connector 35b. The body 35a includes a casing 70 and a fluid transfer part 50 which provides a fluid channel. The fluid transfer part 50 is secured within the casing 70 and retains the cannula 40 within the patient. The fluid transfer part 50 may be integral with the casing 70.

The tube connector 35b engages an interface 45 of the fluid transfer part 50 to connect a downstream end of the tube 15 to the fluid transfer part 50. The tube connector 35b includes a releasable connector, in this case a releasable snap-fit joint.

As shown in FIG. 1B, the tube connector 35b has mechanical elements in the form of resiliently deformable arms 21 that engage correspondingly mechanical receiving elements on the body 35a of the infusion hub 35, or more specifically, the casing 70 of the body 35a. While the mechanical elements are arranged as a releasable clip in the illustrated tube connector 25b, it would be apparent that this is not essential. When the tube connector 35b is secured to the body 35a, a needle 75 of the tube connector 35b pierces a sealing membrane 65 within the fluid transfer part 50, so that the therapeutic agent can flow through the fluid transfer part 50 and into the patient via the cannula 40.

The function of the fluid transfer part 50 is to allow fluid (e.g. a therapeutic agent) to be transferred through the infusion hub 35 (i.e. from the interface 45 of the fluid transfer part 50 to the cannula 40). As can be seen in FIG. 1A, the fluid transfer part 50 provides a fluid flow path that extends firstly approximately parallel to the plane of the skin surface, and then bends back to be approximately perpendicular to the skin surface.

The fluid transfer part 50 has multiple interfaces 45, 80. A sealing membrane 65 seals a first interface 45, and a second sealing membrane 85 is used to seal a second interface 80. While two interfaces 45, 80 are shown, it would be apparent that more than two interfaces may be provided as required. Some or all the interfaces may have a sealing membrane secured therein to prevent egress of therapeutic agent from the fluid transfer part 50 through the respective interface.

The cannula 40 is a substantially tubular member for insertion in, and delivering a therapeutic agent to, an infusion site 60. The therapeutic agent includes insulin or an insulin solution. A proximal end of the cannula 40 is fluidly connected to a source of therapeutic agent, here an infusion pump 25. An opposing, distal end of the cannula 40 is positioned in the infusion site 60, extending to a desired depth to deliver the therapeutic agent. The cannula 40 is any suitable cannula suitable for implantation in a tissue site of a patient, such as a polymeric catheter or metal needle.

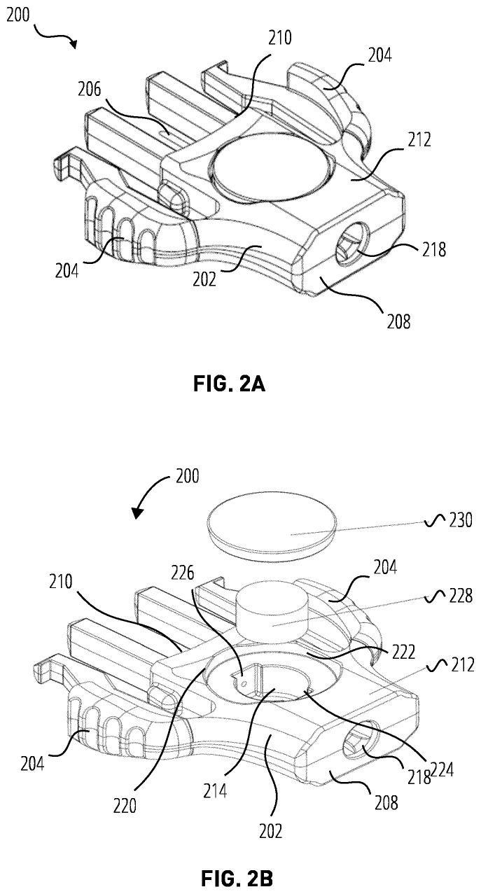

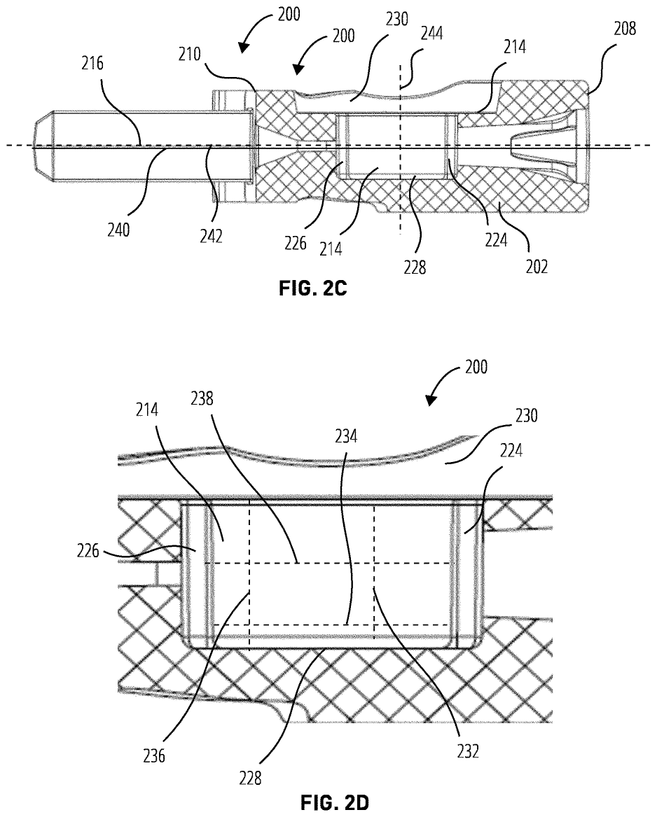

Tube connectors for use with such infusion sets, and in accordance with the present invention, will now be described with particular reference to FIG. 2A to FIG. 2D.

Referring now to FIG. 2A, FIG. 2B, FIG. 2C and FIG. 2D, there is shown a tube connector 200. The tube connector 200 has a body portion 202 which includes mechanical elements, in the form of resiliently deformable arms 204 that engage corresponding mechanical receiving elements on the infusion hub (not shown), and a connector needle 206.

The body portion 202 has a first end 208, a second end 210, an outer surface 212 and a cavity 214. The body portion 202 also has a central axis (a body portion axis 216), as shown in FIG. 2D.

The first end 208 of the body portion 202 is connectable to a tube (for example the tube 15 as shown in FIG. 1A and FIG. 1B) for receiving a therapeutic agent from a pump (for example pump 25 as shown in FIG. 1B). In particular, the first end 208 includes a connection portion 218 that is configured to receive a tube.

The second end 210 of the body portion 202 is connectable to a fluid transfer part (for example the fluid transfer part 50 as shown in FIG. 1A and FIG. 1B) for fluid communication with a cannula of the infusion hub (for example the cannula 40 of the infusion hub 35 as shown in FIG. 1A).

With particular reference to FIG. 2B, the cavity 214 is located between the first end 208 and the second end 210 of the body portion 202.

The outer surface 212 of the body portion 202 includes an opening 220. The opening 220 defines a channel 222 that extends from the outer surface 212 of the body portion 202 to the cavity 214.

The body portion 202 includes a first fluid chamber 224 and a second fluid chamber 226.

The first fluid chamber 224 is positioned between the first end 208 of the body portion 202 and the cavity 214.

The second fluid chamber 226 is positioned between the second end 210 of the body portion 202 and the cavity 214.

As can also be seen in FIG. 2B, the tube connector 200 includes a filter 228 and a cap 230.

The cavity 214 is configured to receive the filter 228. That is, the shape and size of the cavity 214 and the shape and size of the filter 228 is such that, in use, the filter 228 is received within the cavity 214.

In this example, each of the cavity 214 and the filter 228 are generally cylindrical.

The channel 222 is configured to receive the cap 230. That is, the shape and size of the channel 222 corresponds to the shape and size of the cap 230 such that, in use, the cap 230 is received within and seals the channel 222 between the opening 220 and the cavity 214. The cap 230 is press-fit into the channel 222 and sealed by any suitable method, for example laser welding or heat welding. In examples of the invention, a seal may be provided on a surface of the channel 222 and/or the cap 230 in order to ensure that the channel 222 is sealedly closed for use once the filter 228 is positioned within the cavity 214.

With particular reference to FIG. 2D, the cavity 214 has a cavity depth 232 and a cavity width 234 (the cavity width 234 corresponding to the diameter of the cylindrical cavity 214). Similarly, the filter 228 has a filter depth 236 and a filter width 238 (the filter width 238 corresponding to the diameter of the cylindrical filter 228).

Referring now to FIG. 2C, a fluid flow path 240 is defined through the body portion 202.

The fluid flow path 240 extends from the first end 208 of the body portion 202 to the second end 210 of the body portion 202, through the first fluid chamber 224, the cavity 214, the filter 228 and the second fluid chamber 226. The fluid flow path 240 also has a central axis (i.e. a fluid flow path axis 242). The body portion axis 216 and the fluid flow path axis 242 are coaxial.

The cavity 214 has a central axis (i.e. a cavity axis 244). The cavity axis 244 is not coaxial with the body portion axis 216 or the fluid flow path axis 242. The cavity axis 244 is perpendicular to the body portion axis 216 and the fluid flow path axis 242.

As can be seen in FIG. 2D, the filter 228 is sized such that the filter depth 236 is greater than the cavity depth 232 and the filter width 238 is greater than the cavity width 234 such that, when the filter 228 is installed in the cavity 214 and the cap 230 is installed in the channel 222, the filter 228 fills the volume of the cavity 214 of the body portion 202.

It will be appreciated that the filter 228 may be any filter 228 that is suitable for removing, for example by filtration, unwanted species present in the therapeutic agent to circumvent FBR. Used herein, unwanted species is to be understood to mean one or more species which may be present in the therapeutic agent, for example by design or accident, and which may be undesirable to remain in the therapeutic agent at the point of delivery to the infusion site. In particular, the filter may remove unwanted species that occur in insulin solutions. Such unwanted species may be particulate and/or molecular in nature. Examples of particulate unwanted species include plastic particles, dust and insulin agglomerates, which have been produced during manufacture, storage, sterilization, or handling of the infusion set and/or the insulin solution. Examples of molecular unwanted species include preservatives commonly used in insulin solutions, such as phenol, cresol (particularly m-cresol), benzyl alcohol, benzalkonium chloride, cetrimide, chlorobutanol, chlorhexidine, chlorocresol, hydroxybenzoates, phenethyl alcohol, phenoxyethanol and phenylmercuric nitrate.

The filter may be a sintered filter. The filter may comprise one or more of cellulose (for example regenerated cellulose or cellulose acetate), polytetrafluoroethylene, nylon, polyethersulfone, polyvinylidene fluoride, polyethylene and polypropylene.

The filter is disposed within the tube connector and, in particular within a cavity in the body portion of the tube connector. The cavity opening allows insertion of a larger filter (i.e. a filter that has an increased filter area), which advantageously helps to preserve unrestricted flow through the tube connector, whilst filtering unwanted species from the therapeutic agent.

The filter may include any filter material capable of removing one or more unwanted species from the therapeutic agent. To remove particulate unwanted species, the filter material may provide a physical filter medium for removing unwanted species by size exclusion, including whereby the filter material functions as a molecular sieve. Additionally, or alternatively, to remove molecular unwanted species, the filter material may provide a chemical filter medium for removing unwanted species by sorption, for example by adsorption or ion exchange, whereby the filter material binds with the molecular unwanted species to retain them within the filter. The filter material has a plurality of passageways, for example pores (i.e., interconnected hollow voids), extending therethrough to allow fluid flow through the filter.

The filter may be a modular filter including first and second sub-filters arranged to allow fluid flow therethrough in series for progressively removing different unwanted species from the therapeutic agent, for example unwanted species of varying sizes and/or varying molecular composition. Accordingly, the first sub-filter may include a filter material different to that of the second sub-filter. The filter may similarly include a third, a fourth and so on sub-filters.

Suitable filter materials include a foam, for example a polyvinyl alcohol (PVA) foam, and foams made of a cellulose, a polyurethane, a polyester, a polyether, a collagen or the like.

The filter may be a sintered filter, for example a sintered foam filter, a sintered ceramic filter or a sintered membrane filter.

The foam includes a plurality of passageways in the form of interconnected pores extending therethrough to allow fluid flow through the filter. The foam may be any foam capable of removing particulate unwanted species from an insulin solution. The foam may remove particulate unwanted species from an insulin solution by a size exclusion process.

The pores may be millipores (pore size from 0.1 to 100 mm). Suitable foams include those having a pore size from 0.1 to 5 mm. Particularly suitable foams include those having a pore size from 0.3 to 1 mm. Additionally or alternatively, suitable foams may include those having a porosity from 50% to 95% and/or dry density of from 0.1 to 1.5 grams per cubic inch. Particularly suitable foams include those having a porosity from 90% to 95%, and/or for example a dry density of from 0.8 to 1.5 grams per cubic inch. The foam may also absorb an aqueous solution such that the foam is saturated by at least 95% in a time from 0.1 to 1 minutes, for example 3 to 30 seconds. The foam may have an ability to retain a liquid insulin solution such that the weight of the retained insulin solution is from 5 to 100 times, for example from 10 to 25 times, the weight of the foam material in the absence of an insulin solution. The foam may be crosslinked.

While a foam is described herein, it would be apparent other structures, such as a membrane or a sheet or similar may be used in place of a foam and the properties described in relation to foams apply equally to membranes or sheets.

Particularly suitable filter materials include polyvinyl alcohol (PVA) foams, examples of which and methods of making are known in the art, and examples are disclosed in earlier patent applications, including U.S. Pat. No. 4,083,906 and U.S. Ser. No. 11/197,949, the contents of which are incorporated herein by reference.

Suitable filter materials include a zeolite. Zeolites are microporous, aluminosilicate minerals commonly used as commercial adsorbents and catalysts. The zeolite may be any zeolite capable of removing particulate and/or molecular unwanted species from an insulin solution. Most suitably, the zeolite is capable of removing phenol and/or cresol (particularly m-cresol) from an insulin solution. Particulate unwanted species may be removed from an insulin solution by a size exclusion process whereby the zeolite functions as a molecular sieve. Molecular unwanted species may be removed from the insulin infusion mixture by an adsorption process whereby the zeolite binds to the molecular components and retains them within the zeolite framework. Suitable zeolites include those having a pore size from 3 to 10 Å. Particularly suitable zeolites are those having a pore size from 5 to 8 Å. The zeolite may have a FAU, MOR or MFI framework type. The zeolite may have been thermally treated prior to being incorporated into the embodiments of the invention. For example, the zeolite may have been heated to a temperature in excess of 200° C., or even in excess of 700° C., prior to being incorporated into the embodiments of the invention. The zeolite may be coated, for example with a dextran. The zeolite may be a hydrophobic zeolite.

Particularly suitable zeolites include zeolite Y, mordenite and silicalite, for which example methods of preparation are known in the art, and disclosed in “Phenolic Preservative Removal from Commercial Insulin Formulations Reduces Tissue Inflammation while Maintaining Euglycemia”, Adam Mulka et al., ACS Pharmacology & Translational Science 2021 4 (3), 1161-1174, the contents of which is incorporated herein by reference.

Suitable filter materials include an ion-exchange resin, including functionalized porous or gel polymers, which may remove unwanted species from an insulin solution by a gel permeation chromatography process. Moreover, gel polymers may be used to coat passageways in the filter material.

Generally, the filter material may be selected to have at least one material property that may facilitate the infusion of insulin at a single infusion site over an extended period of time, and thereby increase wear times, for example at least four days, including four to seven days, seven or more days, seven to 10 days, 10 or more days, and 14 or more days.

Advantageously, the filter 228 of the tube connector 200 removes, for example by filtration, unwanted species present in the therapeutic agent, thereby circumventing a foreign body reaction (FBR).

In the embodiment described above, the body portion 202 is formed as a single component within which the cavity 214, first fluid chamber, second fluid chamber and fluid flow path are defined (i.e. the body portion 202 has a unitary construction).

It will be appreciated that, in other examples of the invention, the body portion 202 may include a first body portion part and a second body portion part. In this example, the cavity 214, first fluid chamber, second fluid chamber and fluid flow path are defined between the first body portion part and the second body portion part. The first body portion part may form an upper portion of the body portion 202 and the second body portion part may form a lower portion of the body portion 202. Alternatively, the first body portion part may form a lower portion of the body portion 202 and the second body portion part may form an upper portion of the body portion 202. The opening may be formed in the first body portion part or the second body portion part. The opening may be formed in the upper portion or the lower portion of the body portion 202.

In some examples of the invention, an opening may be provided in each of the first body portion part and the second body portion part. An opening may be, for example formed in the upper portion and the lower portion of the body portion 202. In such examples, a first cap is provided to close one of the openings and a second cap is provided to close the other of the openings.

In the embodiment described above, each of the cavity 214 and the filter 228 are generally cylindrical. It will be appreciated that, in other embodiments of the invention, the cavity and the filter may be any shape or size, so long as the filter can be received in the cavity of the body portion, in use.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics or groups described in conjunction with a particular aspect, embodiment or example of the invention are to be understood to be applicable to any other aspect, embodiment or example described herein unless incompatible therewith. All the features disclosed in this specification (including any accompanying claims, abstract and drawings), and/or all the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the steps of any method or process so disclosed.

1. A tube connector for an infusion hub, the tube connector comprising: a body portion comprising: a first end connectable to a tube for receiving a therapeutic agent from a pump; a second end connectable to a fluid transfer part for fluid communication with a cannula of the infusion hub, in use; an outer surface; a fluid flow path defined between the first end and the second end of the body portion; a cavity positioned between the first end and the second end of the body; and a filter, wherein the cavity comprises an opening on the outer surface of the body portion through which the filter is inserted into the cavity.

2. The tube connector of claim 1, wherein the fluid flow path extends along a central axis of the body portion and wherein the cavity comprises a cavity axis that is perpendicular to the central axis.

3. The tube connector of claim 1 or 2, wherein the body portion comprises a first body portion part and a second body portion part and the cavity is defined between the first body portion part and the second body portion part.

4. The tube connector of claim 3, wherein one of the first body portion part and the second body portion part forms an upper portion of the body portion and the other of the first body portion part and the second body portion part forms a lower portion of the body portion.

5. The tube connector of claim 4, wherein the opening is provided in the upper portion of the body portion.

6. The tube connector of claim 4, wherein the opening is provided in the lower portion of the body portion.

7. The tube connector of any one of claims 1 to 6, wherein the body portion comprises a cap that is configured to close the opening, thereby securing the filter in the cavity.

8. The tube connector of any one of claims 1 to 7, wherein a fluid chamber is provided between the first end of the body portion and the cavity.

9. The tube connector of claim 8, wherein the fluid chamber is a first fluid chamber and a second fluid chamber is provided between the cavity and the second end of the body portion.

10. The tube connector of any one of claims 1 to 9, wherein the cavity has a depth and the filter has a depth, wherein the filter depth is greater than the cavity depth.

11. The tube connector of any one of claims 1 to 10, wherein the cavity has a width and the filter has a width, wherein the filter width is greater than the cavity width.

12. The tube connector of any one of claims 1 to 11, wherein the filter comprises cellulose.

13. The tube connector of claim 12, wherein the cellulose is regenerated cellulose or cellulose acetate.

14. The tube connector of any one of claims 1 to 13, wherein the filter comprises one or more of polytetrafluoroethylene, nylon, polyethersulfone, polyvinylidene fluoride, polyethylene and polypropylene.

15. The tube connector of any one of claims 1 to 14, wherein the filter is a sintered filter.

16. An infusion set comprising an infusion hub having a cannula and a mounting portion for mounting the infusion hub to a patient's skin with the cannula in a subcutaneous position, and the tube connector of any one of claims 1 to 15.

17. The infusion set of claim 16, wherein one of the tube connector and the infusion hub comprises a needle.

18. An infusion system comprising the infusion set of claim 16 or claim 17 and a source of medicament for delivery to the patient via the tube connector and the cannula.

19. The infusion system of claim 18, further comprising a pump for continuous or intermittent delivery of medicament to the patient.
